(19) United States
(12) Patent Application Publication
(10) Pub. No.: US 2011/0281901 A1
(43) Pub. Date: Nov. 17, 2011

(54) PHARMACEUTICAL COMPOSITIONS AND METHODS OF MAKING SAME

(75) Inventor: Manish Gupta, Collegeville, PA (US)

(73) Assignee: GLAXO WELLCOME MANUFACTURING PTE LTD., Singapore (SG)

(21) Appl. No.: 13/101,689

(22) Filed: May 5, 2011

Related U.S. Application Data
(60) Provisional application No. 61/331,715, filed on May 5, 2010.

(51) Int. Cl. A61K 31/506; A61P 27/02

(52) U.S. Cl. 514/275

(57) ABSTRACT

Pharmaceutical compositions that include an amount of an acid addition salt of pazopanib equivalent to about 5 mg/mL pazopanib, about 2.0 to about 13.0% w/w of a modified cyclodextrin suitable for use in an ophthalmic formulation, and pH adjusting agent as well as methods of making the same are described.

### CROSS-REFERENCE TO RELATED APPLICATIONS

This application is filed pursuant to 35 U.S.C. §111(a) claiming priority from U.S. Provisional Application No. 61/331,715 filed on May 5, 2010.

### FIELD OF THE INVENTION

This invention relates to pharmaceutical compositions and methods of making the same, particularly pharmaceutical formulations suitable for ocular administration.

### BACKGROUND OF THE INVENTION

Pazopanib is a highly bio-available, multi-tyrosine kinase inhibitor of vascular endothelial growth factor receptor (VEGFR)-1, -2, -3, platelet-derived factor receptor (PDGFR)-α, -β, cytokine receptor (cKit), interleukin-2 receptor inducible T-cell kinase (Itk), leukocyte-specific protein tyrosine kinase (Lck), and transmembrane glycoprotein receptor tyrosine kinase (c-Fms). WO 2007/064752 describes the use of pazopanib to treat age-related macular degeneration. It is desirable to provide a stable eye-drop formulation of pazopanib in which the pazopanib is solubilized in the formulation. Pazopanib is a poorly water-soluble drug, having a solubility in phosphate buffer using pazopanib free base of 0.000006 at pH 5.0.

Cyclodextrins are used in drug formulations as solubility enhancers because of their ability to form water-soluble inclusion complexes with otherwise poorly water-soluble drugs. The fundamental property that describes the strength of interaction between a drug and a cyclodextrin is the binding constant (or stability constant) K. The cyclodextrin utility number (UcD) is a dimensionless number that can be used to assess the feasibility of the use of cyclodextrins in dosage forms. The U_{CD} allows the formulator to determine if the use of cyclodextrins in the formulation of poorly water-soluble drugs has the potential to provide a significant solubilization advantage. U_{CD} is calculated using the following equation:

U_{CD}=(KSₒ/1+KSₒ)(m_{CD}/m_{D})(MW_{D}/MW_{CD})

where:

m_{D} is dose of drug;

m_{CD} is dose of cyclodextrin;

MW_{D} is molecular weight drug;

MW_{CD} is molecular weight cyclodextrin; and

Kₒ is binding constant.

When the dimensionless number U_{CD} is greater than or equal to 1, solubilization is adequately provided by the complexation of cyclodextrins with the drug. When the dimensionless number is less than 1, the complexation alone is not enough for complete solubilization. For ophthalmic formulations, the workable amount of the cyclodextrin, m_{CD}, can depend upon the desired tonicity of the solution. See, V. M. Rao & V. J. Stella, When Can Cyclodextrins Be Considered for Solubilization Purposes?, J. Pharm. Sci., Vol. 92, No. 5 (2003).

It is desirable to provide a stable eye-drop formulation in which an amount of an acid addition salt of pazopanib equivalent to 5 mg/mL pazopanib free base is solubilized in the formulation.

### SUMMARY OF THE INVENTION

In one aspect of the present invention, a pharmaceutical composition includes an amount of an acid addition salt of pazopanib equivalent to about 5 mg/mL pazopanib, about 2.0 to about 13.0% w/w of a modified cyclodextrin suitable for use in an ophthalmic formulation, and pH adjusting agent. The composition is suitable for administration to the eye of a human and has a U_{CD} value in the range of 0.0002 to 0.6 at a temperature of 25° C.

In another aspect of the present invention, a pharmaceutical composition includes an amount of an acid addition salt of pazopanib equivalent to about 5 mg/mL pazopanib, about 2.0 to about 13.0% w/w of a modified cyclodextrin suitable for use in an ophthalmic formulation, and pH adjusting agent, where the composition is suitable for administration to the eye of a human and is a super-saturated aqueous solution of pazopanib.

In still another aspect of the present invention, a method of preparation of a super-saturated composition includes forming an aqueous solution of an acid addition salt of pazopanib and a modified cyclodextrin suitable for use in an ophthalmic formulation, and adjusting the pH of said solution to between about 4 to about 5 to obtain a super-saturated solution of pazopanib.

### BRIEF DESCRIPTION OF THE SEVERAL VIEWS OF THE DRAWING

FIG. 1 illustrates a flow diagram of the manufacturing process of pazopanib hydrochloride solution.

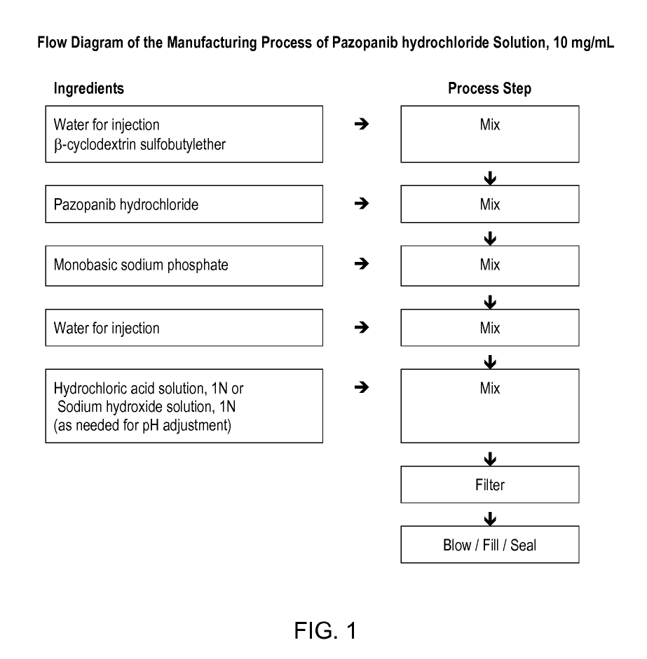

### DETAILED DESCRIPTION OF THE INVENTION

As used herein, “super-saturated solution” means a solution containing more solute than would a saturated solution under given conditions of temperature and pressure.

As used herein, the chemical name “pazopanib” refers to the compound 5-[[4-[(2,3-dimethyl-2H-indazol-6-yl)methylamino]-2-pyrimidinyl]amino]-2-methylbenzenesulfonamide, which compound is represented by Structure I:

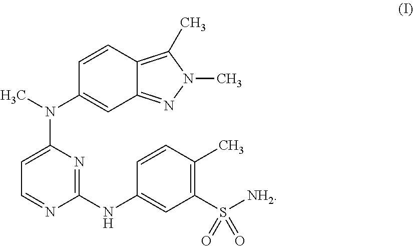

In some embodiments according to the various aspects of the present invention described herein, the acid addition salt of the compound of formula (I) is a hydrochloride salt. In a particular embodiment, the acid addition salt of the compound of formula (I) is a monohydrochloride salt as illustrated by formula (I′). The monohydrochloride salt of the compound of formula (I) has the chemical name 5-[[4-[(2,3-dimethyl-2H-indazol-6-yl)methylamino]-2-pyrimidinyl]amino]-2-methylbenzenesulfonamide monohydrochloride.

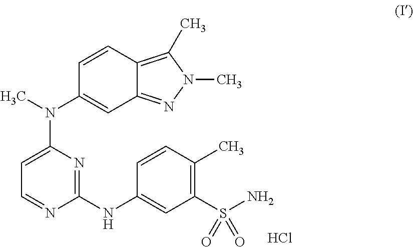

In other embodiments, the acid addition salt of the compound of formula (I) is a monohydrochloride monohydrate solvate of the compound of formula (I). The monohydrochloride monohydrate solvate of the compound of formula (I) has the chemical name 5-({4-[(2,3-dimethyl-2H-indazol-6-yl)methylamino]-2-pyrimidinyl} amino)-2-methylbenzenesulfonamide monohydrochloride monohydrate, as illustrated in formula (I″).

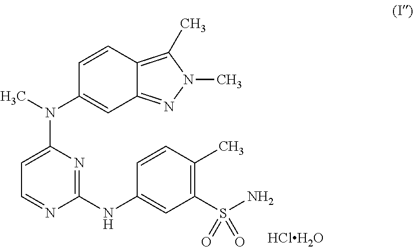

The free base, salts and solvates of the compound of formula (I) may be prepared, for example, according to the procedures of International Patent Application No. PCT/US01/49367 filed Dec. 19, 2001, and published as WO 02/059110 on Aug. 1, 2002, and International Patent Application No. PCT/US03/19211 filed Jun. 17, 2003, and published as WO 03/106416 on Dec. 24, 2003, or according the methods provided herein.

As used herein, the term “acid addition salts” are salts derived from a nitrogen on a substituent in the compound of formula (I). Representative salts include the following salts: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylbromide, methylnitrate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, tannate, tartrate, teoclate, tosylate, triethiodide, trimethylammonium and valerate.

Pazopanib is a poorly water-soluble drug, having an approximate solubility (mg/mL) at 25° C. in phosphate buffer using pazopanib free base as follows: 0.000006 at pH 5.0, 0.000025 mg/mL at pH 4.5; 0.000534 at pH 4.25; 0.001043 mg/mL at pH 4.0; and 0.02 at pH 3.5.

In one aspect of the present invention, a pharmaceutical composition includes an amount of an acid addition salt of pazopanib equivalent to about 5 mg/mL pazopanib, about 2.0 to about 13.0% w/w of a modified cyclodextrin suitable for use in an ophthalmic formulation, and pH adjusting agent. The composition is suitable for administration to the eye of a human and has a UcD value in the range of 0.0002 to 0.6 at a temperature of 25° C.

The cyclodextrin utility number, U_{CD}, is a dimensionless number used to assess the feasibility of using cyclodextrin in dosage forms. U_{CD} is a lumped parameter consisting of the dose of the drug, the workable amount of CD, the binding constant, and the drug solubility in the absence of CDs. U_{CD} can be used to predict the solubility of ionizable drugs showing a synergistic increase in solubility due to ionization and complexation. See, Rao, V. M., Stella, V. J., J Pharm Sci, 92, 5 927, May 2003. U_{CD} is calculated using the following equation:

U_{CD}=(KSₒ/1+KSₒ)(m_{CD}/m_{D})(MW_{D}/MW_{CD})

where,
m_{D} is dose of drug;
m_{CD} is dose of cyclodextrin;
MW_{D} is molecular weight drug;
MW_{CD} is molecular weight cyclodextrin; and
Kₒ is binding constant.

In some embodiments, the U_{CD} value is in the range from a lower limit of about 0.0002, 0.0003, 0.0004, 0.0005, 0.0006, 0.0007, 0.0008, 0.0009, 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.105, 0.11, 0.115, 0.12, 0.125, 0.13, 0.135, 0.14, 0.145, 0.15, 0.155, 0.16, 0.165, 0.17, 0.175, 0.18, 0.185, 0.19, 0.195, 0.2, 0.205, 0.21, 0.215, 0.22, 0.225, 0.23, 0.235, 0.24, 0.245, 0.25, 0.255, 0.26, 0.265, 0.27, 0.275, 0.28, 0.285, 0.29, 0.295, 0.3, 0.305, 0.31, 0.315, 0.32, 0.325, 0.33, 0.335, 0.34, 0.345, 0.35, 0.355, 0.36, 0.365, 0.37, 0.375, 0.38, 0.385, 0.39, 0.395, or 0.4 and an upper limit of about 0.2, 0.205, 0.21, 0.215, 0.22, 0.225, 0.23, 0.235, 0.24, 0.245, 0.25, 0.255, 0.26, 0.265, 0.27, 0.275, 0.28, 0.285, 0.29, 0.295, 0.3, 0.305, 0.31, 0.315, 0.32, 0.325, 0.33, 0.335, 0.34, 0.345, 0.35, 0.355, 0.36, 0.365, 0.37, 0.375, 0.38, 0.385, 0.39, 0.395, 0.4, 0.405, 0.41, 0.415, 0.42, 0.425, 0.43, 0.435, 0.44, 0.445, 0.45, 0.455, 0.46, 0.465, 0.47, 0.475, 0.48, 0.485, 0.49, 0.495, 0.5, 0.505, 0.51, 0.515, 0.52, 0.525, 0.53, 0.535, 0.54, 0.545, 0.55, 0.555, 0.56, 0.565, 0.57, 0.575, 0.58, 0.585, 0.59, 0.595, or 0.6.

In another aspect of the present invention, a pharmaceutical composition includes an amount of an acid addition salt of pazopanib equivalent to about 5 mg/mL pazopanib, about 2.0 to about 13.0% w/w of a modified cyclodextrin suitable for use in an ophthalmic formulation, and pH adjusting agent, where the composition is suitable for administration to the eye of a human and is a super-saturated aqueous solution of pazopanib.

In still another aspect of the present invention, a method of preparation of a super-saturated composition includes forming an aqueous solution of an acid addition salt of pazopanib and a modified cyclodextrin suitable for use in an ophthalmic formulation, and adjusting the pH of said solution to between about 4 to about 5 to obtain a super-saturated solution of pazopanib.

A modified cyclodextrin that is suitable for use in an ophthalmic formlation is a modified cyclodextrin that is tolerated in the human eye to an extent that the formulation can be administered to a human at least once per day for at least one month. In some embodiments according to the various aspects of the present invention described herein, the modified cyclodextrin is selected from the group consisting of hydroxypropyl-β-cyclodextrin, methyl-β-cyclodextrin, β-cyclodextrin sulfobutylether and combinations thereof. In other embodiments, the modified cyclodextrin is β-cyclodextrin sulfobutylether.

In some embodiments according to the various aspects of the present invention described herein, the amount of the modified cyclodextrin is in the range of a lower limit of about 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, or 9.0% w/w to an upper limit of about 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9 or 13.0% w/w.

In some embodiments according to the various aspects of the present invention described herein, the osmolality of the composition is in the range of a lower limit of about 200, 210, 220, 230, 240, 250, 260, 270, 280, 290, 300, 310, 320, 330, 340, or 350 mOsm to an upper limit of about 250, 260, 270, 280, 290, 300, 310, 320, 330, 340, 350, 360, 370, 380, 390, or 400 mOsm.

Some embodiments according to the various aspects of the present invention described herein, the composition further includes one or more agents selected from tonicity adjusting agents and/or buffering agents. The tonicity adjusting agents and/or buffering agents may be any of various such agents known to those of skill in the art to be suitable for inclusion in a composition for ocular administration to the human eye. In some embodiments, the tonicity adjusting agents and/or buffering agents are selected from the group consisting of sodium chloride, sodium phosphate, and combinations thereof. In some embodiments, the tonicity adjusting agent and/or buffering agent is sodium chloride. In some embodiments, the amount of sodium chloride is in the range of a lower limit of 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 mM to an upper limit of about 30, 31, 32, 33, 34, 35, 36, 37, 38, 39, 40, 41, 42, 43, 44, 45, 46, 47, 48, 49 or 50 mM. In other embodiments, the tonicity adjusting agent and/or buffering agent is sodium phosphate. In some embodiments, the amount of sodium phosphate is in the range of a lower limit of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 mM to an upper limit of about 30, 31, 32, 33, 34, 35, 36, 37, 38, 39, 40, 41, 42, 43, 44, 45, 46, 47, 48, 49 or 50 mM.

The pH adjusting agent may be various such agents known to those of skill in the art to be suitable for inclusion in a composition for ocular administration to the human eye. In some embodiments according to the various aspects of the present invention, the pH adjusting agent is selected from the group consisting of sodium hydroxide, hydrochloric acid and combinations thereof.

In some embodiments according to the various aspects of the present invention described herein, the pH of the composition is in the range of a lower limit of about 3.5, 3.55, 3.6, 3.65, 3.7, 3.75, 3.8, 3.85, 3.9, 3.95, 4.0, 4.05, 4.1, 4.15, 4.2, 4.25, 4.3, 4.35, 4.4, 4.45, 4.5, 4.55, 4.6, 4.65, 4.7, 4.75, 4.8, 4.85, 4.9, 4.95, 5.0, 5.05, 5.1, 5.15, 5.2, 5.25, 5.3, 5.35, 5.4, 5.45, 5.5, 5.55 or 5.6 to an upper limit of about 5.0, 5.05, 5.1, 5.15, 5.2, 5.25, 5.3, 5.35, 5.4, 5.45, 5.5, 5.55, 5.6, 5.65, 5.7, 5.75, 5.8, 5.85, 5.9, 5.95, or 6.0. In some embodiments according to the various aspects of the present invention described herein, the composition is physically stable for 2, 6, 12 or 24 months as measured according to USP <789>“Particulate Matter in Opthalmic Solutions.”

The following examples are intended for illustration only and are not intended to limit the scope of the invention in any way.

### EXAMPLES

### Example 1

### Composition

Compositions of pazopanib monohydrochloride solution are given below in Table 1.

TABLE 1

| Component | 10 mg/mL | 2 mg/mL | 5 mg/mL | Function |
|---|---|---|---|---|
| Pazopanib | 10.834 | 2.167 | 5.417 | Active ingredient |
| hydrochloride | | | | pazopanib in the |
| | | | | form of the |
| | | | | monohydrochloride |
| | | | | salt |
| β-cyclodextrin | 90.000 | 70.000 | 70.000 | Solubility enhancer |
| Sulfobutylether | | | | |
| Monobasic | 3.450 | 3.450 | 3.450 | Buffering agent |
| Sodium | | | | |
| Phosphate | | | | |
| Water for | q.s. | 1.685 | 1.461 | Solvent |
| Injection | | | | |
| Sodium | q.s. | q.s. | q.s. | pH adjustment |
| Hydroxide | | | | |
| Hydrochloric | q.s. | q.s. | q.s. | pH adjustment |
| Acid | | q.s. | q.s. | |

### Example 2

### Stability Data

The physical stability of pazopanib monohydrochloride solution, 10 mg/mL, pH 4.0 is given in Table 2.

TABLE 2

Test

| | | Pazopanib | |
|---|---|---|---|
| | | hydrochloride | |
| | | Content | |
| Storage | Time | (% label | |
| Condition | (Months) | claim) | pH |
| Initial | 0 | 104.3 | 4.2 |
| 5° C. | 1 | 104.1 | 4.1 |
| | 3 | 104.1 | 4.0 |
| 25° C./60% RH | 1 | 103.7 | 4.1 |
| | 3 | 104.4 | 4.0 |
| 40° C./25% RH | 1 | 104.6 | 4.1 |
| | 3 | 105.2 | 4.0 |

### Example 3

### Method of Preparing 10 mg/mL Composition Pazopanib Monohydrochloride Solution

Manufacture of the bulk solution is conducted in a Grade C environment. β-cyclodextrin sulfobutylether is added to an appropriate vessel containing water for injection (WFI) and gently mixed until visibly dissolved. The following ingredients are then individually added to the vessel in order, gently mixed and allowed to dissolve before proceeding to the next addition: active ingredient (pazopanib in the form of the monohydrochloride salt), and monobasic sodium phosphate, monohydrate. The solution is brought to volume with WFI and gently mixed. The pH of the solution is adjusted to 4.25, if necessary, using 1 N hydrochloric acid or 1 N sodium hydroxide solution. The resulting solution is filtered using two sterile 0.22 μm sterilizing filters (in series), followed by a third 0.22 μm filter within the blow-fill-seal equipment, prior to filling.

### Example 4

### Calculation of U_{CD}

The UCD calculation for a pazopanib monohydrochloride solution is given below in Table 3.

TABLE 3

| | Pazopanib | |
|---|---|---|
| | monohydrochloride | |
| | calculations, pH 4.5 | |
| Dose of drug, m_{D} | 0.4 | mg |
| Dose of cyclodextrin, | 3.6 | mg |
| as β-cyclodextrin | | |
| sulfobutylether, m_{CD} | | |
| MW_{D} | 437.5 | |
| MW_{CD} | 2200 | |
| Assuming Kₒ to be | 10000 | L/mol |
| (KSₒ)/(1 + KSₒ) | 0.012058531 | |
| [m_{CD}/m_{D}] | 9 | |
| [MW_{D}/MW_{CD}] | 0.198863636 | |
| U_{CD} | 0.0215820 | |

U_{CD}=(KSₒ/1+KSₒ)(m_{CD}/m_{D})(MW_{D}/MW_{CD})

Dose of drug, m_{D}
Dose of cyclodextrin, m_{CD}
Molecular weight drug, MW_{D}
Molecular weight cyclodextrin, MW_{CD}
Binding constant, Kₒ
from Rao, V. M., Stella, V. J., J Pharm Sci, 92, 5 927, May 2003.
The UCD calculation for a 5 mg/mLpazopanib monohydrochloride and 7% Captisol® (β-cyclodextrin sulfobutylether) solution at various pH levels is given below in Table 4.

TABLE 4

| | pH 3.5 | pH 4.0 | pH 4.25 | pH 4.5 | pH 5.0 |
|---|---|---|---|---|---|
| Dose of drug, m_{D} (mg) | 0.2 | 0.2 | 0.2 | 0.2 | 0.2 |
| Dose of cyclodextrin, | 2.8 | 2.8 | 2.8 | 2.8 | 2.8 |
| as β-cyclodextrin | | | | | |
| sulfobutylether, | | | | | |
| m_{CD} (mg) | | | | | |
| MW_{D} | 437.5 | 437.5 | 437.5 | 437.5 | 437.5 |
| MW_{CD} | 2200 | 2200 | 2200 | 2200 | 2200 |
| Kₒ (assumed) (L/mol) | 10000 | 10000 | 10000 | 10000 | 10000 |
| (KSₒ)/(1 + KSₒ) | 0.314 | 0.023 | 0.012 | 0.0006 | 0.0001 |
| [m_{CD}/m_{D}] | 14 | 14 | 14 | 14 | 14 |
| [MW_{D}/MW_{CD}] | 0.199 | 0.199 | 0.199 | 0.199 | 0.199 |
| U_{CD} | 0.87 | 0.065 | 0.034 | 0.0016 | 0.0004 |

The UCD calculation for a5 mg/mLpazopanib monohydrochloride and 12% Captisol® (β-cyclodextrin sulfobutylether) solution at various pH levels is given below in Table 5.

TABLE 5

| | pH 3.5 | pH 4.0 | pH 4.25 | pH 4.5 | pH 5.0 |
|---|---|---|---|---|---|
| Dose of drug, m_{D} (mg) | 0.2 | 0.2 | 0.2 | 0.2 | 0.2 |
| Dose of cyclodextrin, | 4.8 | 4.8 | 4.8 | 4.8 | 4.8 |
| as β-cyclodextrin | | | | | |
| sulfobutylether, | | | | | |
| m_{CD} (mg) | | | | | |
| MW_{D} | 437.5 | 437.5 | 437.5 | 437.5 | 437.5 |
| MW_{CD} | 2200 | 2200 | 2200 | 2200 | 2200 |
| Kₒ (assumed) (L/mol) | 10000 | 10000 | 10000 | 10000 | 10000 |
| (KSₒ)/(1 + KSₒ) | 0.314 | 0.023 | 0.012 | 0.0006 | 0.0001 |
| [m_{CD}/m_{D}] | 24 | 24 | 24 | 24 | 24 |
| [MW_{D}/MW_{CD}] | 0.199 | 0.199 | 0.199 | 0.199 | 0.199 |
| U_{CD} | 1.5 | 0.11 | 0.058 | 0.0027 | 0.0007 |

The UCD calculation for a 5 mg/mLpazopanib monohydrochloride and 2% Captisol® (β-cyclodextrin sulfobutylether) solution at various pH levels is given below in Table 6.

TABLE 6

| | pH 3.5 | pH 4.0 | pH 4.25 | pH 4.5 | pH 5.0 |
|---|---|---|---|---|---|
| Dose of drug, m_{D} (mg) | 0.2 | 0.2 | 0.2 | 0.2 | 0.2 |
| Dose of cyclodextrin, | 0.8 | 0.8 | 0.8 | 0.8 | 0.8 |
| as β-cyclodextrin | | | | | |
| sulfobutylether, | | | | | |
| m_{CD} (mg) | | | | | |
| MW_{D} | 437.5 | 437.5 | 437.5 | 437.5 | 437.5 |
| MW_{CD} | 2200 | 2200 | 2200 | 2200 | 2200 |
| Kₒ (assumed) (L/mol) | 10000 | 10000 | 10000 | 10000 | 10000 |
| (KSₒ)/(1 + KSₒ) | 0.314 | 0.023 | 0.012 | 0.0006 | 0.0001 |
| [m_{CD}/m_{D}] | 4 | 4 | 4 | 4 | 4 |
| [MW_{D}/MW_{CD}] | 0.199 | 0.199 | 0.199 | 0.199 | 0.199 |
| U_{CD} | 0.25 | 0.019 | 0.0096 | 0.0005 | 0.0001 |

As described in the Background with reference to the Rao article, the U_{CD} allows the formulator to determine if the use of cyclodextrins in the formulation of poorly water-soluble drugs has the potential to provide a significant solubilization advantage. When the U_{CD} dimensionless number is less than 1, the complexation alone is not enough for complete solubilization. In view of the low U_{CD} values for the 5 mg/mL compositions, it would have been unexpected that compositions such as these would have exhibited the stability necessary for use as clinical trial formulations.

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

What is claimed is:

1. A pharmaceutical composition comprising:
an amount of an acid addition salt of pazopanib equivalent to about 5 mg/mL pazopanib;
about 2.0 to about 13.0% w/w of a modified cyclodextrin suitable for use in an ophthalmic formulation; and
pH adjusting agent;
wherein the composition is suitable for administration to the eye of a human, and wherein the composition has a U_{CD} value in the range of 0.0002 to 0.6 at a temperature of 25° C.

2. The composition of claim 1, wherein the modified cyclodextrin is selected from the group consisting of hydroxypropyl-β-cyclodextrin, methyl-β-cyclodextrin, β-cyclodextrin sulfobutylether and combinations thereof.

3. The composition of claim 1, wherein the modified cyclodextrin is β-cyclodextrin sulfobutylether.

4. The composition of claim 1, wherein the amount of the modified cyclodextrin is in the range of about 6% to about 11% w/w.

5. The composition of claim 1, wherein the osmolality of the composition is in the range of about 200 to about 400 mOsm.

6. The composition of claim 1, wherein the osmolality of the composition is in the range of about 250 to about 350 mOsm.

7. The composition of claim 1, further comprising one or more agents selected from tonicity adjusting agents and buffering agents.

8. The composition of claim 7, wherein said tonicity adjusting agents and buffering agents are selected from the group consisting of sodium chloride, sodium phosphate, and combinations thereof.

9. The composition of claim 7, wherein at least one of said tonicity adjusting agents and buffering agents is sodium chloride, and wherein the amount of sodium chloride is in the range of 0 to about 50 mM.

10. The composition of claim 7, wherein at least one of said tonicity adjusting agents and buffering agents is sodium phosphate, and wherein the sodium phosphate is in the range of about 10 to about 50 mM.

11. The composition of claim 1, wherein the pH adjusting agent is selected from the group consisting of sodium hydroxide, hydrochloric acid and combinations thereof.

12. The composition of claim 1, wherein the pH of said ophthalmic composition is in the range of about 4 to about 5.

13. The composition of claim 1, wherein the pH of said ophthalmic composition is in the range of about 4.5 to about 5.5.

14. The composition of claim 1, wherein the composition is physically stable as measured according to USP <789>“Particulate Matter in Opthalmic Solutions at 5° C. for at least 2 months.

15. The composition of claim 1, wherein the composition is physically stable as measured according to USP <789>“Particulate Matter in Opthalmic Solutions at 5° C. for at least 6 months.

16. The composition of claim 1, wherein the composition is physically stable as measured according to USP <789>“Particulate Matter in Opthalmic Solutions at 5° C. for at least 1 year.

17. The composition of claim 1, wherein the composition is physically stable as measured according to USP <789>“Particulate Matter in Opthalmic Solutions at 5° C. for at least 2 years.

18. The composition of claim 1, wherein the acid addition salt of pazopanib is pazopanib hydrochloride.

19. A pharmaceutical composition comprising:
an amount of an acid addition salt of pazopanib equivalent to about 5 mg/mL pazopanib;
about 2.0 to about 13.0% w/w of a modified cyclodextrin suitable for use in an ophthalmic formulation; and
pH adjusting agent;
wherein the composition is suitable for administration to the eye of a human, and wherein the composition is a super-saturated aqueous solution of pazopanib.

20. The composition of claim 19, wherein the modified cyclodextrin is selected from the group consisting of hydroxypropyl-β-cyclodextrin, methyl-β-cyclodextrin, β-cyclodextrin sulfobutylether and combinations thereof.

21. The composition of claim 19, wherein the modified cyclodextrin is β-cyclodextrin sulfobutylether.

22. The composition of claim 19, wherein the amount of the modified cyclodextrin is in the range of about 6% to about 11% w/w.

23. The composition of claim 19, wherein the osmolality of said composition is in the range of about 200 to about 400 mOsm.

24. The composition of claim 19, wherein the osmolality of the composition is in the range of about 250 to about 350 mOsm.

25. The composition of claim 19, further comprising one or more buffering agents and tonicity agents.

26. The composition of claim 25, wherein the one or more tonicity adjusting and buffering agents are selected from the group consisting of sodium chloride, sodium phosphate, and combinations thereof.

27. The composition of claim 25, wherein at least one of the tonicity adjusting agents is sodium chloride, and wherein the amount of sodium chloride is in the range of 0 to about 50 mM.

28. The composition of claim 25, wherein at least one of the buffering agents is sodium phosphate, and wherein the sodium phosphate is in the range of about 10 to about 50 mM.

29. The composition of claim 25, wherein the pH adjusting agent is selected from the group consisting of sodium hydroxide, hydrochloric acid and combinations thereof.

30. The composition of claim 19, wherein the pH of the ophthalmic composition is in the range of about 4 to about 5.

31. The composition of claim 19, wherein the pH of the ophthalmic composition is in the range of about 4.5 to about 5.5.

32. The composition of claim 19, wherein the composition is physically stable as measured according to USP <789>“Particulate Matter in Opthalmic Solutions at 5° C. for at least 2 months.

33. The composition of claim 19, wherein the composition is physically stable as measured according to USP <789>“Particulate Matter in Opthalmic Solutions at 5° C. for at least 6 months.

34. The composition of claim 19, wherein the composition is physically stable as measured according to USP <789>“Particulate Matter in Opthalmic Solutions at 5° C. for at least 1 year.

35. The composition of claim 19, wherein the composition is physically stable as measured according to USP <789>“Particulate Matter in Opthalmic Solutions at 5° C. for at least 2 years.

36. The composition of claim 19, wherein the acid addition salt of pazopanib is pazopanib hydrochloride.

37. A method of preparation of a super-saturated solution of pazopanib, said method comprising:
forming an aqueous solution of an acid addition salt of pazopanib that is equivalent to about 5 mg/mL of pazopanib and a modified cyclodextrin suitable for use in an ophthalmic formulation; and
adjusting the pH of said solution to between about 4 to about 5 to obtain a super-saturated solution of pazopanib.

38. The method of claim 37, wherein the acid addition salt of pazopanib is pazopanib hydrochloride.

39. The method of claim 37, wherein the modified cyclodextrin is selected from the group consisting of hydroxypropyl-β-cyclodextrin, methyl-β-cyclodextrin, β-cyclodextrin sulfobutylether and combinations thereof.

40. The method of claim 37, wherein the modified cyclodextrin is β-cyclodextrin sulfobutylether.

41. The method of claim 37, wherein the amount of the modified cyclodextrin is in the range of about 2.0% to about 13.0% w/w.

42. The method of claim 37, wherein the amount of the modified cyclodextrin is in the range of about 6% to about 11% w/w.
